(19) United States
(12) Patent Application Publication
(10) Pub. No.: US 2003/0013116 A1
(43) Pub. Date: Jan. 16, 2003

(54) Multiplex protein interaction determinations using glutathione-GST binding

(75) Inventors: Yong Song, San Pablo, CA (US); Quan Nguyen, Pleasant Hill, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA

(21) Appl. No.: 10/243,099

(22) Filed: Sep. 13, 2002

Related U.S. Application Data
(62) Division of application No. 09/792,404, filed on Feb. 22, 2001.

(51) Int. Cl. C12Q 1/68; G01N 33/53; G01N 33/542; G01N 33/537; G01N 33/543

(52) U.S. Cl. 435/6; 435/7.5; 435/7.92

(57) ABSTRACT

Fusion proteins in which glutathione S-transferase (GST) is a fusion partner are used as an immobilized binding member in screening procedures or other multi-analyte test procedures based on protein interaction. In such procedures, a particular protein is selected from a group of candidate proteins on the basis of the binding affinity of that protein for a target protein, with either the candidate proteins or the target protein being a fusion partner with GST and the GST portion of the fusion partner having been immobilized on glutathione-coated particles by the binding of GST to glutathione. The particles themselves are classifiable by different values of a differentiation parameter that permits them to be distinguished by flow cytometry, and the procedure is conducted in a manner that associates the individual candidate proteins with individual classes of the particles. When a binding interaction occurs between a candidate protein and the target protein, the particles on which the interaction has occurred are readily distinguished by flow cytometry and correlated with the candidate protein that exhibited the binding.

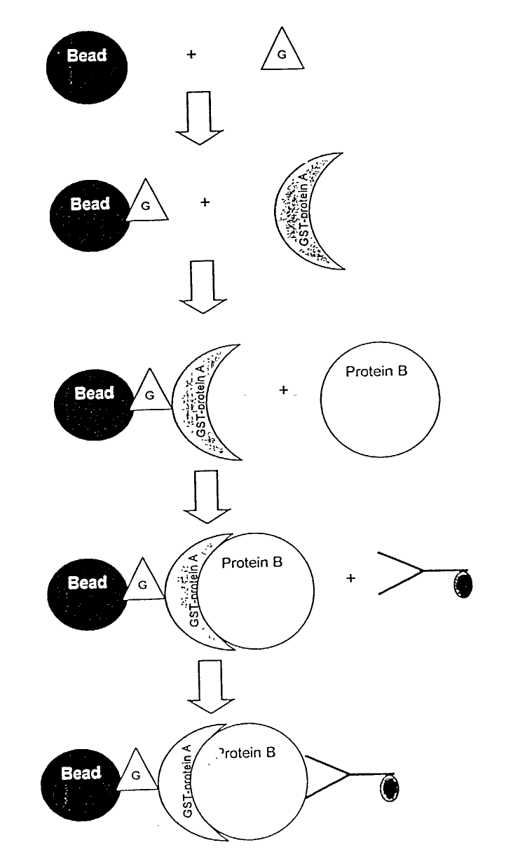

### BACKGROUND OF THE INVENTION

1. Field of the Invention

This invention resides in the field of screening assays to assess protein-protein interactions, and to screen candidate proteins for their affinity to target proteins.

2. Description of the Prior Art

Many clinical and research investigations involve the study of protein-protein interactions for purposes such as screening proteins or peptides to find those that display binding specificity to a particular protein, determining the binding affinity of two interacting proteins, and identifying the site or amino acid of a protein that is responsible for the interaction between that protein and a second protein. Information relevant to the function of a protein can be obtained by determining whether and how that protein interacts with another protein of known function. This type of information is also of value in the design and screening of drugs, and generally in developing methods for the diagnosis and treatment of diseases.

Of further relevance to this invention is the known specific binding interaction between glutathione and glutathione S-transferase. Glutathione (γ-glutamylcysteinylglycine) is a triamino acid peptide that is found in the cells of higher animals at a concentration of approximately 5 mM. A characteristic feature of glutathione is its linkage at the γ-carboxyl group rather than the α-carboxyl group of the glutamyl residue. Glutathione S-transferase (“GST”) is a 26-kDa protein with a very high affinity for glutathione. Use has been made of this affinity in the purification or proteins, by first forming a recombinant protein in which GST is included as a fusion partner and then purifying the recombinant protein by affinity chromatography on immobilized glutathione columns. GST-containing recombinant proteins have also been used as a means of detecting antibodies to the protein that is fused to GST. These methods are described for example by Murray, A. M., et al., “Production of glutathione-coated microtitre plates for capturing recombinant glutathione S-transferase fusion proteins as antigens in immunoassays,” J. Immunol. Meth. 218 (1998): 133-139.

Of further possible relevance to this invention is the state of the art relating to the use of flow cytometry for the detection and analysis of particles and species bound to microparticles. Flow cytometry has been disclosed for use in the detection and separation of antigens and antibodies by Coulter Electronics Inc., United Kingdom Patent No. 1,561,042 (published Feb. 13, 1980); and for quantitation of PCR (Polymerase Chain Reaction) products by Vlieger, A. M., et al., Analytical Biochemistry 205:1-7 (1992). The use of magnetic particles in flow cytometry is disclosed in International Patent Application Publication No. WO99/26067, “Multiplex Flow Immunoassays With Magnetic Particles as Solid Phase,” of applicant Bio-Rad Laboratories, Inc., published May 27, 1999. All references listed above are incorporated herein by reference.

### SUMMARY OF THE INVENTION

It has now been discovered that glutathione-GST binding can serve effectively as an immobilizing linkage in studying protein-protein interactions using differentiable groups of solid particles in a multiplex format. The present invention thus resides in a variety of screening or selection methods in which a particular protein is selected from a group of candidate proteins by virtue of the binding affinity of the selected protein toward a target binding member such as another protein, the selection occurring by flow cytometry or other methods of differentiating particles.

In one aspect of the invention, fusion proteins are formed, each including one candidate protein as a fusion partner with GST. Once formed, the fusion proteins are immobilized on glutathione-coated particles of different groups or classes that can be differentiated from each other by flow cytometry, each group having a different fusion protein and hence a different candidate protein such that individual candidate proteins are associated with separately differentiable particle classes. All particles are then incubated with the binding member to which the desired candidate protein will selectively bind, and the particles to which the binding member has become bound are detected. By correlating the detected particle class with the candidate protein that is included in the fusion protein bound to that particle class, one can identify the candidate protein that demonstrates specific binding to the target species. The same or a similar method can be used to compare binding affinities (i.e., different binding strengths) among different candidate proteins, by detecting differences in the proportion of the target species that binds to each class of particles.

In another aspect, a fusion protein is formed by combining GST with the target binding member. Additional fusion proteins are then formed, each one containing one candidate protein plus an epitope tag. The GST-containing fusion protein is then immobilized on glutathione-coated particles of different groups or classes that can be differentiated from each other by flow cytometry, and each class is then incubated with one of the candidate protein-epitope fusion proteins. Each particle class will then have been incubated with a distinct candidate protein-epitope fusion protein and hence a distinct candidate protein, and only those particles that have been incubated with candidate proteins that selectively bind to the target binding member will then bear the epitope (through the various affinity-binding and covalent linkages). The presence of the epitope tag on these particles can then be determined by incubating the particles with labeled antibody to the epitope tag, and correlation and identification can be performed as described above.

In a third aspect, the candidate proteins are conjugated to a directly detectable label such as a fluorescent label, and the conjugates are used in place of the candidate protein-epitope fusion proteins.

Additional aspects, embodiments, implementations, and applications of the central concepts of this invention will become apparent from the description that follows.

### BRIEF DESCRIPTION OF THE DRAWING

The FIGURE is a symbolic representation of a screening procedure in accordance with this invention.

### DETAILED DESCRIPTION OF THE INVENTION AND SPECIFIC EMBODIMENTS

The term “fusion protein” is used herein to denote a combination of two proteins or peptides joined in any manner or by any type of linkage, covalent, electrostatic, hydrophobic-interaction, affinity-type, or otherwise, that maintains the linkage between the partners, prevents cleavage of the linkage during the procedural steps that are followed in the practice of this invention, and leaves the binding characteristics of the protein substantially unchanged. A preferred kind of fusion protein for the purpose of this invention is a polypeptide made from a recombinant gene that contains portions of two or more different genes, the genes being joined so that their coding sequences are in the same reading frame, i.e., so that the genetic apparatus reads the gene fusion as a single gene. This type of fusion protein is also known as a hybrid protein or a chimeric protein.

The term “conjugate” is used herein in connection with proteins to denote a combination of a protein and another species, such as a label, a binding member, or an epitope tag, joined by any type of linkage, covalent, electrostatic, hydrophobic-interaction, affinity-type, or otherwise, in a manner that will maintain the integrity of the linkage and prevent it from cleavage during the procedural steps that are followed in the practice of this invention, and leave the binding characteristics of the protein substantially unchanged.

The term “candidate protein” is used herein to denote a polypeptide of any length or size that is to be compared with other polypeptides in terms of binding specificity, affinity or both.

Glutathione-coated particles for use in this invention are prepared by methods known in the art. Suitable methods are those in which the glutathione is coupled to the particle surface in such a manner that the binding affinity of glutathione to GST is unimpaired. Such a coupling may be achieved for example at the central sulfhydryl group of glutathione. These sulfhydryl groups can be covalently joined to lysine residues on a protein coating that has previously been applied to the particle surface. Thus, a preferred means of forming glutathione-coated particles is to first coat the particle with a protein that exhibits a very low (or zero) level of non-specific affinity toward GST and that has lysine residues that will remain accessible for coupling, and then coupling the sulfhydryl groups of the glutathione molecules to the lysine residues through a heterobifunctional crosslinking agent. An example of a protein with low non-specific binding affinity toward GST and with accessible lysine groups is hemoglobin. An example of a heterobifunctional crosslinking agent suitable for coupling the lysine residues and the sulfhydryl groups is sulfosuccinimidyl 4-(p-maleimidophenyl)butyrate. Other examples of both lysine-containing proteins and heterobifunctional crosslinking agents will be readily apparent to those skilled in the art.

Using hemoglobin and sulfosuccinimidyl 4-(p-maleimidophenyl)butyrate, the coating procedure may for example consist of incubating the particles for several hours with a 2% (by weight) solution of bovine hemoglobin in 0.05 M sodium carbonate at pH 9.6, washing the particles, then incubating the particles with a solution of sulfosuccinimidyl 4-(p-maleimidophenyl)butyrate (0.1-1.0 mM) in PBS at room temperature for one hour. The particles are then washed with PBS, and incubated with a solution of reduced glutathione (10-50 mM) in degassed 10 mM sodium phosphate, 0.15 M sodium chloride, 1 mM ethylenediamine tetraacetic acid, pH 6.7, for several hours at room temperature, followed by washing.

Recombinant methods for preparing fusion proteins are well known to those skilled in the art, and such known procedures can be used in the practice of this invention. The procedure may for example consist of forming a construct of the coding region of the protein to be fused with GST (i.e., either the candidate protein or the target binding protein, depending on the protocol) and that of GST and inserting the construct into the frame of the GST fusion protein expression vector (for example, pGEX-5x-1, obtainable from Amersham Pharmacia Biotech, Piscataway, N.J., USA), and then expressing the protein in E. Coli. Expression of the protein (i.e., the candidate protein or the target protein) can then be detected by Western blot analysis with anti-GST antibody.

Incubations for the binding of the GST portions of the fusion proteins to the glutathione coatings on the particles and for the binding of the candidate proteins to the target binding members can be conducted by routine procedures with which those skilled in immunology and protein binding studies are well familiar. The separation of solid phase from liquid phase and the washing steps are likewise performed in accordance with conventional and routine techniques.

Detection of the candidate protein-target protein binding is accomplished by the use of any of the wide variety of labels that are known to be effective in immunoassays and other procedures in which affinity-type binding or protein detection in general occurs. The label may be conjugated to an antibody to the target protein, for example, when the GST fusion proteins are fusions of GST and the candidate proteins. The label in this instance may for example be a fluorescent label, a chemiluminescent label, or any other label that emits a signal that is detectable and measurable by automated instrumentation. Alternatively, a biotinylated antibody can be used, and detection accomplished by incubating the particles with fluorophore-labeled avidin. A particularly convenient fluorophore for this type of use is phycoerythrin. As a further alternative, the candidate proteins or target protein may be conjugated to a detectable label such as a fluorophore or a chemiluminescent label. Thus, when the GST fusion proteins are fusions of GST and the candidate proteins, a target protein that is conjugated to a fluorophore or a chemiluminescent label may be used, and the label detected directly, or when the GST fusion proteins are fusions of GST and the target protein, the individual candidate proteins can be conjugated to a fluorophore or a chemiluminescent label.

The particles used in the practice of this invention are preferably microscopic in size, and therefore may be referred to as microparticles. The microparticles are generally formed of a polymeric material that bears certain characteristics that allow the particles to function effectively in immunoassays. One such characteristic is that the polymeric material be inert to the candidate proteins and target proteins and to the assay reagents other than the reagents used to apply the glutathione coating to the particles. Other characteristics are that the particles be solid and insoluble in the reaction media and in any other solvents or carriers used in the procedure, and that the particles be capable of coupling glutathione to their surface, although this may be achieved by using an intermediate protein coating such as hemoglobin, as described above. When fluorescence will be used as the means of detection, the polymeric material is preferably one that exhibits minimal autofluorescence. Examples of suitable polymers are polyesters, polyethers, polyolefins, polyalkylene oxides, polyamides, polyurethanes, polysaccharides, celluloses, and polyisoprenes. Crosslinking is useful in many polymers for imparting structural integrity and rigidity to the particle.

In embodiments in which detection is performed by fluorescence combined with flow cytometry, care should be taken to avoid the use of particles that emit high autofluorescence since this will interfere with the screening detection. Particles of low autofluorescence can be created by standard emulsion polymerization techniques from a wide variety of starting monomers. Particles of high porosity and surface area (i.e., “macroporous” particles), as well as particles with a high percentage of divinylbenzene monomer, should be avoided since they tend to exhibit high autofluorescence. Generally, however, particles suitable for use in this invention can vary widely in size, and the sizes are not critical to this invention. In most cases, best results will be obtained with particle populations whose particles range from about 0.3 micrometers to about 100 micrometers, preferably from about 0.5 micrometers to about 40 micrometers, in diameter.

In steps of the procedure when the particles are separated from the liquid reaction media, one means of accomplishing such separation is to use particles that are made of or that include a magnetically responsive material. Magnetically responsive materials that can be used in the practice of this invention include paramagnetic materials, ferromagnetic materials, ferrimagnetic materials, and metamagnetic materials. Paramagnetic materials are preferred. Examples are iron, nickel, and cobalt, as well as metal oxides such as Fe₃O₄, BaFe₁₂O₁₉, CoO, NiO, Mn₂O₃, Cr₂O₃, and CoMnP. The magnetically responsive material may constitute the entire particle, but is preferably only one component of the particle, the remainder being a polymeric material to which the magnetically responsive material is affixed.

When particles containing magnetically responsive material are used, the quantity of such material in the particle is not critical and can vary over a wide range. The quantity can affect the density of the particle, however, and both the quantity and the particle size can affect the ease of maintaining the particle in suspension. Maintaining suspension serves to promote maximal contact between the liquid and solid phase and to facilitate flow cytometry. In procedures in which fluorescence plays a role in the detection, an excessive quantity of magnetically responsive material in the particles will also produce autofluorescence at a level high enough to interfere with the procedure. It is therefore preferred that the concentration of magnetically responsive material be low enough to minimize any autofluorescence emanating from the material. With these considerations in mind, the magnetically responsive material in a particle in accordance with this invention preferably ranges from about 1% to about 75% by weight of the particle as a whole. A more preferred weight percent range is from about 2% to about 50%, a still more preferred weight percent range is from about 3% to about 25%, and an even more preferred weight percent range is from about 5% to about 15%. The magnetically responsive material can be dispersed throughout the polymer, applied as a coating on the polymer surface or as one of two or more coatings on the surface, or incorporated or affixed in any other manner that secures the material in the polymer matrix.

Multiplexing with the use of particles in accordance with this invention is achieved by assigning the particles to two or more groups, each group capable of being differentiated from the other group(s) by a “differentiation parameter,” which term is used herein to denote a distinguishable characteristic that permits separate detection of the assay result in one group from that in another group. One example of a differentiation parameter that can be used to distinguish among the various groups of particles is the particle size. The groups in this example are defined by nonoverlapping subranges of size. The particles fall into two or more such subranges, and in most cases the subranges will number from two to 100, each selectively active in a single assay and inert relative to the other assays simultaneously being performed or detected.

The widths of the size subranges and the spacing between mean diameters of adjacent subranges are selected to permit differentiation of the subranges by flow cytometry, and will be readily apparent to those skilled in the use of and instrumentation for flow cytometry. In this specification, the term “mean diameter” refers to a number average diameter. In most cases, a preferred subrange width is about ±5% CV or less of the mean diameter, where CV is the coefficient of variation and is defined as the standard deviation of the particle diameter divided by the mean particle diameter times 100 percent. The minimum spacing between mean diameters among the various subranges can vary depending on the particle size distribution, the ease of segregating particles by size for purposes of attaching different fusion proteins, and the type and sensitivity of the flow cytometry equipment. In most cases, best results will be achieved when the mean diameters of different subranges are spaced apart by at least about 6% of the mean diameter of one of the subranges, preferably at least about 8% and most preferably at least about 10%. Another preferred subrange width relation is that in which the standard deviation of the particle diameters within each subrange is less than one third of the separation of the mean diameters of adjacent subranges.

Another example of a differentiation parameter that can be used to distinguish among the various groups of particles is fluorescence. Differentiation is accomplished by incorporating various fluorescent materials in the particles, the various fluorescent materials having different fluorescence emission spectra and being distinguishable on this basis.

Fluorescence can in fact be used both as a means of distinguishing the groups from each other and as a means of detection for the assay performed on the particle. The use of fluorescent materials with different emission spectra provides a means of distinguishing the groups from each other and as a means of distinguishing the group classification from the assay detections. An example of a combination of fluorescent substances in which one of the substances can be used as a means of distinguishing groups and the other for the assay detection is fluorescein and phycoerythrin. Different particle groups are dyed with differing concentrations of fluorescein and the labeled binding proteins have phycoerythrin coupled thereto as the label.

Still other examples of a differentiation parameter that can be used to distinguish among the various groups of particles are light scatter, light emission, or combinations of light scatter and emission. Side angle light scatter varies with particle size, granularity, absorbance and surface roughness, while forward angle light scatter is mainly affected by size and refractive index. Thus, varying any of these qualities can serve as a means of distinguishing the various groups. Light emission can be varied by incorporating fluorescent materials in the microparticles and using fluorescent materials that have different fluorescence intensities or that emit fluorescence at different wavelengths, or by varying the amount of fluorescent material incorporated. By using a plurality of fluorescent emissions at various wavelengths, the wavelength difference can be used to distinguish the particle groups from each other and also to distinguish the labels indicating the occurrence of binding reactions in the assay from the labels that identify the particle groups.

In a preferred embodiment, the particles will have two or more fluorophores or fluorochromes incorporated within them so that each particle in the array will have at least three distinguishable parameters associated with it, i.e., side scatter together with fluorescent emissions at two separate wavelengths. For example, the particle can be made to contain a red fluorochrome such as Cy5 together with an orange fluorochrome such as Cy5.5. Additional fluorochromes can be used to further expand the system. Each particle can thus contain a plurality of fluorescent dyes at varying wavelengths.

Still another example of a differentiation parameter that can be used to distinguish among the various groups of particles is absorbance. When light is applied to particles the absorbance of the light by the particles is indicated mostly by the strength of the laterally (side-angle) scattered light while the strength of the forward-scattered light is relatively unaffected. Consequently, the difference in absorbance between various colored dyes associated with the particles is determined by observing differences in the strength of the laterally scattered light.

As the above examples illustrate, many different parameters or characteristics can be used as differentiation parameters to distinguish the particles of one group from those of another. The differentiation parameter may arise from particle size, from particle composition, from particle physical characteristics that affect light scattering, from excitable fluorescent dyes or colored dyes that impart different emission spectra and/or scattering characteristics to the particles, or from different concentrations of one or more fluorescent dyes. When the distinguishable particle parameter is a fluorescent dye or color, it can be coated on the surface of the particle, embedded in the particle, or bound to the molecules of the particle material. Thus, fluorescent particles can be manufactured by combining the polymer material with the fluorescent dye, or by impregnating the particle with the dye. Particles with dyes already incorporated and thereby suitable for use in the present invention are commercially available, from suppliers such as Spherotech, Inc. (Libertyville, Ill., USA) and Molecular Probes, Inc. (Eugene, Oreg., USA).

For embodiments of the invention that ail the use of flow cytometry, methods of and instrumentation for flow cytometry are known in the art. Examples of descriptions of flow cytometry instrumentation and methods in the literature are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973). The disclosures in these references are incorporated herein by reference.

The FIGURE is a highly simplified illustration of the binding sequence for a selected procedure in accordance with this invention. A large number of microscopic beads that are divided into individual groups that are distinguishable by flow cytometry (represented in the FIGURE by a single bead in the form of a small circle) are all coated with glutathione (represented by a triangle), which is then allowed to bind to a GST fusion protein (represented by a crescent). In one embodiment of the invention, each fusion protein is a fusion between GST and one of the candidate proteins (shown as “protein A”), and the individual candidate proteins are matched with individual groups of particles. Thus, while only a single crescent symbol is shown in the FIGURE, this represents a plurality of fusion proteins differing from one another by the “protein A” component. A liquid solution of the target protein (which is a single protein and is represented by the larger circle bearing the words “protein B”) is then incubated with the particles of all groups, which no longer need be kept separate but can form a single suspension in the liquid solution, and the target protein will then bind to the fusion protein that includes a candidate protein that has binding affinity toward the target protein. Labeled antibody (represented by the sideways Y-shaped symbol with an attached oval-shaped label) with specific binding affinity toward the target protein (“protein B”) is then incubated with the particles, and binds to the target protein. The particles are then separated from the liquid phase, and those that had a fusion protein attached whose candidate protein component binds to the target protein now have a label adhering to them. These labeled particles are then detected by flow cytometry, and the detected particle group is then correlated with the candidate protein (“protein A”) on that group, thereby identifying the particular candidate protein that binds to the target protein (“protein B”).

As an illustration of the alternative method, the target protein is “protein A” rather than “protein B,” and the fusion protein (the crescent) that is bound to the particles through the glutathione-GST interaction is a common fusion protein (containing the common target protein component) bound to all particles. “Protein B” represents the candidate proteins (a plurality collectively represented by a single symbol), and the individual groups of particles are kept separate through their incubation with the various candidate proteins so that each candidate protein is associated with a single group of particles. The remainder of the procedure is the same as that described in the preceding paragraph, except that the candidate proteins can all be labeled directly and only those that become bound to the particles will have their labels detected. Using the same type of correlation, the particular candidate protein, that binds to the target protein (which is part of the GST fusion protein) is identified.

The foregoing descriptions are offered primarily for purposes of illustration. Further modifications and alternatives of the materials and procedures expressed that are still within the scope of this invention above will be readily apparent to those skilled in the art.

What is claimed is:

1. A method for selecting from among a plurality of candidate proteins those that engage in affinity-type binding with a selected binding member, said method comprising:
(a) forming fusion proteins each comprising one of said candidate proteins fused with glutathione S-transferase;
(b) immobilizing said fusion proteins on a plurality of glutathione-coated particles by affinity between said glutathione S-transferase and said glutathione, said particles classifiable into groups differing by the value of a selected differentiation parameter, such that each group has a different fusion protein bonded thereto;
(c) combining said plurality of particles into a single mixture and incubating said mixture with said selected binding member; and
(d) detecting particles to which said selected binding member has become bound and, by correlating the differentiation parameter value of said particles thus detected with the fusion protein bound thereto, identifying candidate proteins that have bonded to said selected binding member through affinity-type binding.

2. A method in accordance with claim 1 in which said fusion proteins are formed by recombinant DNA.

3. A method in accordance with claim 1 in which step (c) comprises suspending said particles in a liquid medium containing said proteins, and step (d) comprises recovering said particles from said mixture and incubating said recovered particles with labeled antibody to said binding member.

4. A method in accordance with claim 1 in which step (c) comprises suspending said particles in a liquid medium containing said proteins, and step (d) comprises recovering said particles from said mixture, incubating said recovered particles with biotinylated antibody to said binding member, and detecting particles bearing to which biotinylated antibody has become bound by contacting said particles with fluorescent-labeled avidin.

5. A method in accordance with claim 1 in which said glutathione-coated particles comprise particles initially coated with a protein containing an accessible lysine residue, said lysine residue having been covalently linked to the sulfhydryl group of glutathione.

6. A method in accordance with claim 5 in which said protein containing an accessible lysine residue is hemoglobin.

7. A method in accordance with claim 1 in which said differentiation parameter is a member selected from the group consisting of particle size, fluorescence decay time, degree of light scatter, intensity of fluorescence, absorbance, and combinations of forward light scatter, lateral light scatter, and fluorescence intensity at a combination of wavelengths.

8. A method in accordance with claim 1 in which said differentiation parameter is a member selected from the group consisting of fluorescence decay time, intensity of fluorescence, absorbance, and combinations of forward light scatter, lateral light scatter, and fluorescence intensity at a combination of wavelengths.

9. A method for selecting from among a plurality of candidate proteins those that engage in affinity-type binding with a selected binding member, said method comprising:
(a) forming a first fusion protein comprising said selected binding member fused with glutathione S-transferase;
(b) forming a plurality of second fusion proteins each comprising one of said candidate proteins fused with an epitope tag;
(c) immobilizing said first fusion protein on a plurality of glutathione-coated particles by affinity binding between the glutathione S-transferase of said first fusion protein and said glutathione, said particles classifiable into groups differing by the value of a selected differentiation parameter;
(d) incubating each group of particles individually with one of said second fusion proteins, such that a different candidate protein is incubated with each group of particles; and
(e) incubating all of said groups of particles with labeled binding member that binds selectively to said epitope tag, and detecting particles to which said labeled binding member has become bound and, by correlating the differentiation parameter value of said particles thus detected with the second fusion protein bound thereto, identifying candidate proteins that have bonded to said selected binding member through affinity-type binding.

10. A method for selecting from among a plurality of candidate proteins those that engage in affinity-type binding with a selected binding member, said method comprising:
(a) forming a fusion protein comprising said selected binding member fused with glutathione S-transferase;
(b) conjugating each of said candidate proteins with a fluorescent label to form a plurality of fluorescent conjugates;
(c) immobilizing said fusion protein on a plurality of glutathione-coated particles by affinity binding between the glutathione S-transferase of said fusion protein and said glutathione, said particles classifiable into groups differing by the value of a selected differentiation parameter;
(d) incubating each group of particles individually with one of said fluorescent conjugates, such that a fluorescent conjugate of a different candidate protein is incubated with each group of particles; and
(e) detecting particles to which said fluorescent label has become bound and, by correlating the differentiation parameter value of said particles thus detected with the fluorescent conjugate bound thereto, identifying candidate proteins that have bonded to said selected binding member through affinity-type binding.
